(19) United States
(12) Patent Application Publication
(10) Pub. No.: US 2024/0016364 A1
(43) Pub. Date: Jan. 18, 2024

(54) SURGERY SYSTEM, SURGERY CONTROL DEVICE, CONTROL METHOD, AND PROGRAM

(71) Applicant: SONY GROUP CORPORATION, TOKYO (JP)

(72) Inventors: AKIHIKO NAKATANI, TOKYO (JP); SHUNSUKE HAYASHI, TOKYO (JP); MASASHI NAITO, TOKYO (JP); YUKI OGAWA, TOKYO (JP); KAZUTERU GENBA, TOKYO (JP)

(21) Appl. No.: 18/253,840

(22) PCT Filed: Nov. 16, 2021
(86) PCT No.: PCT/JP2021/042004

§ 371 Date: May 22, 2023

(30) Foreign Application Priority Data
Nov. 30, 2020 (JP) 2020-198246

(51) Int. Cl. A61B 1/00; A61B 34/00

(52) U.S. Cl. CPC A61B 1/00006; A61B 1/00052; A61B 34/25; A61B 2034/254; A61B 2017/00203

(57) ABSTRACT

The present technique relates to a surgery system, a surgery control device, a control method, and a program that allow a practitioner to easily operate a device during surgery. A surgery region is captured to acquire a surgery image that is an image of the surgery region, and a display image is output on the basis of the surgery image. With at least one of a surgical instrument and a hand reflected on the surgery image as an operation body, a predetermined instruction operation by the operation body is accepted on the basis of an image of the operation body in the surgery image. The present technique is applied to an endoscope system and a microscope surgery system.

### TECHNICAL FIELD

The present technique relates to a surgery system, a surgery control device, a control method, and a program, and in particular to a surgery system, a surgery control device, a control method, and a program that allow a practitioner to easily operate a device during surgery.

### BACKGROUND ART

PTL 1 discloses a user interface through which medical equipment can be operated by voice recognition and visual line recognition without the use of hands.

### CITATION LIST

### Patent Literature

### [PTL 1]

JP-2018-161377A

### SUMMARY

### Technical Problem

A practitioner may use both hands in surgery, and it is not easy to both operate an imaging device for photographing a surgery region or the like and perform surgery. In comparison with an operation by hands, an operation by voice or the like without the use of hands is not easy because it is not intuitive.

The present technique has been made in view of such a situation and allows a practitioner to easily operate a device during surgery.

### Solution to Problem

A surgery system according to a first aspect of the present technique is a surgery system including a medical imaging device that captures a surgery region to acquire a surgery image that is an image of the surgery region, and a control device that outputs a display image to a display device on a basis of the surgery image acquired by the medical imaging device, in which, with at least one of a surgical instrument and a hand reflected on the surgery image as an operation body, the control device accepts a predetermined instruction operation by the operation body related to control of the medical imaging device or the control device on the basis of an image of the operation body in the surgery image.

In the first aspect of the present technique, the surgery region is captured to acquire the surgery image that is the image of the surgery region, and the display image is output on the basis of the surgery image. With at least one of a surgical instrument and a hand reflected on the surgery image as an operation body, a predetermined instruction operation by the operation body is accepted on the basis of an image of the operation body in the surgery image.

A surgery control device or a program according to a second aspect of the present technique is a surgery control device including a processing unit that accepts, with at least one of a surgical instrument and a hand reflected on a surgery image obtained by photographing a surgery region as an operation body, a predetermined instruction operation by the operation body on the basis of an image of the operation body in the surgery image, or a program that allows a computer to function as such a surgery control device.

A control method according to the second aspect of the present technique is a control method in which a surgery control device has a processing unit and the processing unit accepts, with at least one of a surgical instrument and a hand reflected on a surgery image obtained by photographing a surgery region as an operation body, a predetermined instruction operation by the operation body on the basis of an image of the operation body in the surgery image.

In the second aspect of the present technique, with at least one of a surgical instrument and a hand reflected on a surgery image obtained by photographing a surgery region as an operation body, a predetermined instruction operation by the operation body is accepted on the basis of an image of the operation body in the surgery image.

### BRIEF DESCRIPTION OF DRAWINGS

FIG. 1 is a diagram depicting an example of a schematic configuration of an endoscope system.

FIG. 2 is a block diagram depicting an example of functional configurations of a camera head and a CCU depicted in FIG. 1.

FIG. 3 is a diagram depicting an example of a schematic configuration of a microscope surgery system.

FIG. 4 is a diagram depicting the CCU of FIG. 1 in functional blocks.

FIG. 5 is a diagram exemplifying a display image in which a virtual menu is displayed.

FIG. 6 is a diagram exemplifying a display image in which the virtual menu is displayed.

FIG. 7 is diagram exemplifying a display image in which the virtual menu is superimposed on a non-important region.

FIG. 8 is diagram exemplifying a display image in which the virtual menu is superimposed on the non-important region.

FIG. 9 is a diagram exemplifying a display image when a predetermined button of the virtual menu in FIG. 8 is in a button selection state.

FIG. 10 is a flowchart exemplifying a procedure of processing related to the presentation of the virtual menu performed by the CCU.

FIG. 11 is a block diagram depicting a configuration example of hardware of a computer that executes a series of processing by a program.

### DESCRIPTION OF EMBODIMENT

Hereinafter, an embodiment of the present technique will be described with reference to the drawings.

### <Embodiment of Endoscope System to Which Present Technique Is Applied>

The technique according to the present disclosure is applicable to a surgery system (medical imaging system). The surgery system is a medical system using an imaging technique, and is, for example, an endoscope system, a microscope surgery system, or the like.

### [Endoscope System]

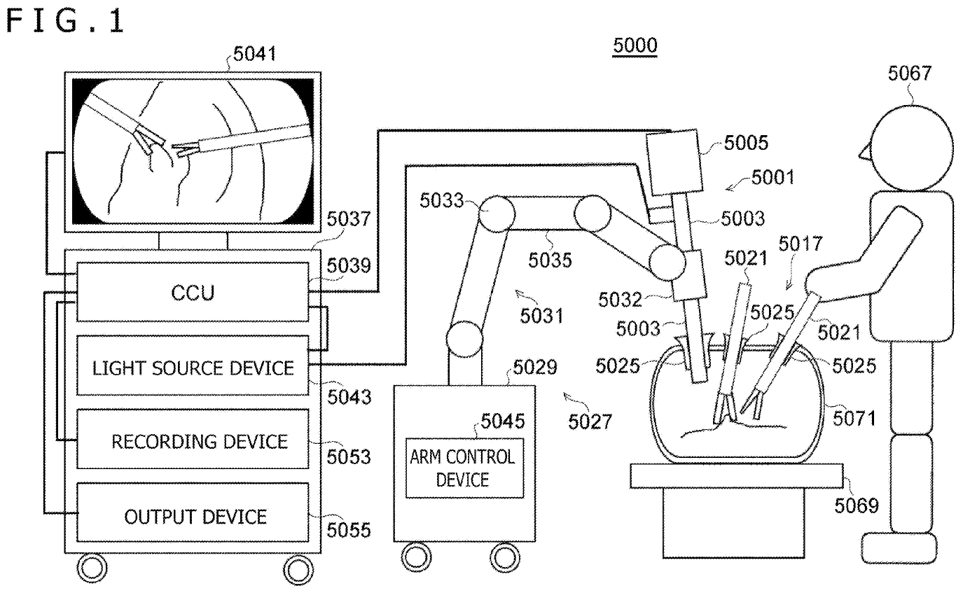

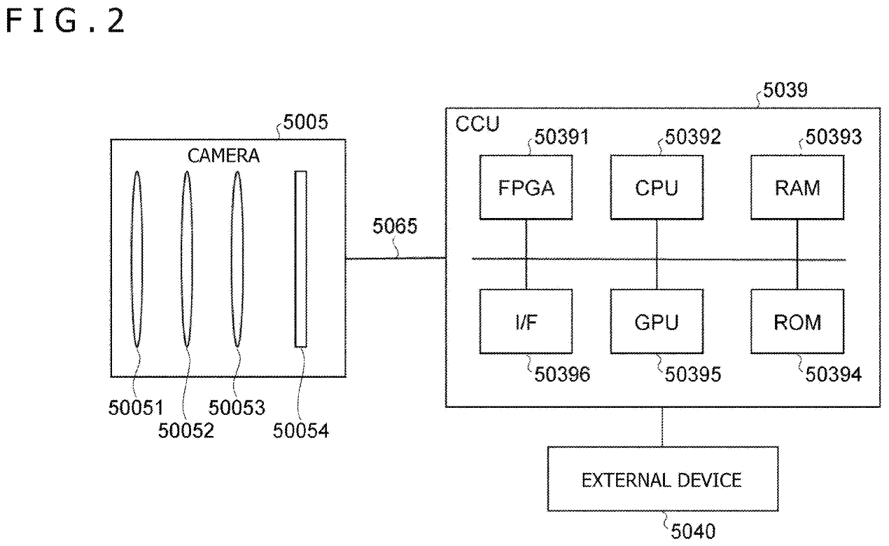

An example of an endoscope system will be described using FIG. 1 and FIG. 2. FIG. 1 is a diagram for depicting an example of a schematic configuration of an endoscope system 5000 to which the technique of the present disclosure is applicable. FIG. 2 is a diagram for depicting an example of a configuration of an endoscope 5001 and a CCU (Camera Control Unit) 5039. FIG. 1 illustrates a state in which a practitioner (for example, a doctor) 5067 who is a surgical participant is performing surgery on a patient 5071 on a patient bed 5069 by using the endoscope system 5000. As depicted in FIG. 1, the endoscope system 5000 is configured using the endoscope 5001 that is a medical imaging device, the CCU 5039, a light source device 5043, a recording device 5053, an output device 5055, and a support device 5027 that supports the endoscope 5001.

In the endoscopic surgery, insertion aids called trocars 5025 are punctured in the patient 5071. Then, a scope 5003 connected to the endoscope 5001 and surgical instruments 5021 are inserted into the inside of the body of the patient 5071 via the trocars 5025. The surgical instruments 5021 are, for example, an energy device such as an electric scalpel and forceps.

A surgical image that is a medical image reflecting the inside of the body of the patient 5071 photographed by the endoscope 5001 is displayed on a display device 5041. The practitioner 5067 performs a procedure on a surgical target by using the surgical instruments 5021 while viewing the surgical image displayed on the display device 5041. It should be noted that the medical image is not limited to the surgical image, but may be a diagnostic image captured during diagnosis.

### [Endoscope]

The endoscope 5001 is a camera that images the inside of the body of the patient 5071, and is a camera head that includes, for example, a light condensing optical system 50051 that condenses incident light, a zoom optical system 50052 that enables optical zoom by changing the focal distance of the camera, a focus optical system 50053 that enables focus adjustment by changing the focal distance of the camera, and a light receiving element 50054, as depicted in FIG. 2. The endoscope 5001 generates a pixel signal by condensing light to the light receiving element 50054 via the connected scope 5003, and outputs the pixel signal to the CCU 5039 through a transmission system. It should be noted that the scope 5003 is an insertion part that has an objective lens at the distal end thereof and guides light from the connected light source device 5043 into the inside of the body of the patient 5071. The scope 5003 is, for example, a rigid scope in a rigid mirror and a flexible scope in a flexible mirror. In addition, the pixel signal may be a signal based on a signal output from a pixel, and is, for example, a RAW signal or an image signal. In addition, a memory may be mounted in the transmission system connecting the endoscope 5001 and the CCU 5039 to each other to store parameters related to the endoscope 5001 and the CCU 5039 into the memory. The memory may be arranged, for example, at a connection part of the transmission system or on a cable. For example, parameters at the time of the shipment of the endoscope 5001 or parameters changed during energization may be stored in the memory of the transmission system to change the operation of the endoscope on the basis of the parameters read from the memory. In addition, a set of the endoscope and the transmission system may also be referred to as an endoscope. The light receiving element 50054 is a sensor that converts the received light into the pixel signal, and is, for example, a CMOS (Complementary Metal Oxide Semiconductor) type imaging element. The light receiving element 50054 is preferably an imaging element that has a Bayer array and enables color photography. In addition, the light receiving element 50054 is preferably an imaging element having the number of pixels corresponding to a resolution of, for example, 4K (3840 horizontal pixels×2160 vertical pixels), 8K (7680 horizontal pixels×4320 vertical pixels), or square 4K (3840 horizontal pixels or more×3840 vertical pixels or more). The light receiving element 50054 may be a single sensor chip or a plurality of sensor chips. For example, a prism for separating incident light for each predetermined wavelength band may be provided to capture each wavelength band with a different light receiving element. In addition, a plurality of light receiving elements may be provided for stereoscopic vision. In addition, the light receiving element 50054 may be a sensor including an arithmetic processing circuit for image processing in a chip structure, or a sensor for ToF (Time of Flight). It should be noted that the transmission system is, for example, an optical fiber cable or wireless transmission. It is only necessary for the wireless transmission to be capable of transmitting the pixel signal generated by the endoscope 5001, and, for example, the endoscope 5001 and the CCU 5039 may be wirelessly connected to each other, or the endoscope 5001 and the CCU 5039 may be connected to each other via a base station in the surgical room. At this time, the endoscope 5001 may simultaneously transmit not only the pixel signal but also information (for example, processing priority of the pixel signal, a synchronization signal, or the like) related to the pixel signal. It should be noted that the scope and the camera head may be integrated into the endoscope, or the light receiving element may be provided at the distal end part of the scope.

### [CCU (Camera Control Unit)]

The CCU 5039 is a control device that comprehensively controls the connected endoscope 5001 and light source device 5043, and is, for example, an information processing device having an FPGA 50391, a CPU 50392, a RAM 50393, a ROM 50394, a GPU 50395, and an I/F 50396, as depicted in FIG. 2. In addition, the CCU 5039 may also comprehensively control the connected display device 5041, the recording device 5053, and the output device 5055. For example, the CCU 5039 controls the irradiation timing and irradiation intensity of the light source device 5043 and the type of irradiation light source. In addition, the CCU 5039 performs image processing such as development processing (for example, demosaic processing) and correction processing on the pixel signal output from the endoscope 5001, and outputs the processed pixel signal (for example, an image) to an external device such as the display device 5041. In addition, the CCU 5039 transmits a control signal to the endoscope 5001 to control the driving of the endoscope 5001. The control signal is, for example, information related to capturing conditions such as the magnification and the focal distance of the camera. It should be noted that the CCU 5039 may have an image down-conversion function and be configured to be capable of simultaneously outputting a high-resolution (for example, 4K) image to the display device 5041 and a low-resolution (for example, HD) image to the recording device 5053.

In addition, the CCU 5039 may be connected to external equipment via an IP converter that converts a signal into a predetermined communication protocol (for example, an IP (Internet Protocol)). The connection between the IP converter and the external equipment may be configured with use of a wired network, or a part or all of the network may be built with use of a wireless network. For example, the IP converter on the CCU 5039 side may have a wireless communication function to transmit the received video to an IP switcher or an output-side IP converter via a wireless communication network such as a 5th generation mobile communication system (5G) or a 6th generation mobile communication system (6G).

### [Light Source Device]

The light source device 5043 is a device capable of applying light having a predetermined wavelength band and includes, for example, a plurality of light sources and a light source optical system for guiding light beams of the plurality of light sources. The light source is, for example, a xenon lamp, an LED light source, or an LD light source. The light source device 5043 has LED light sources corresponding to, for example, three primary colors R, G, and B, and emits white light by controlling the output intensity and output timing of each light source. In addition, the light source device 5043 may have a light source capable of applying special light used for special light observation, separately from a light source for applying normal light used for normal light observation. The special light is light having a predetermined wavelength band different from normal light that is light for normal light observation, and is, for example, near-infrared light (light having a wavelength of 760 nm or more), infrared light, blue light, or ultraviolet light. The normal light is, for example, white light or green light. In narrow-band light observation that is a kind of special light observation, by alternately applying blue light and green light, it is possible to photograph predetermined tissues, such as blood vessels in the surface layer of the mucous membrane, at high contrast by utilizing the wavelength dependency of light absorption in the body tissues. In addition, in fluorescence observation that is a kind of special light observation, by applying excitation light that excites a medicine injected into the body tissues and receiving the fluorescence emitted by the body tissues or the medicine that is a marker to obtain a fluorescence image, it is possible for the practitioner to easily see the body tissues and the like that are difficult for the practitioner to see by normal light. For example, in infrared light observation using infrared light, by applying near-infrared light as excitation light that excites a medicine such as indocyanine green (ICG) injected into the body tissues, it is possible to easily see the structure in the back of the body tissues. In addition, in fluorescence observation, a medicine (for example, 5-ALA) that is excited by special light having a blue wavelength band and emits fluorescence having a red wavelength band may be used. These medicines are photodynamic therapy, and it should be noted that the type of irradiation light of the light source device 5043 is set under the control of the CCU 5039. The CCU 5039 may have a mode in which the normal light observation and the special light observation are alternately performed by controlling the light source device 5043 and the endoscope 5001. At this time, it is preferable that information based on the pixel signal obtained by the special light observation is superimposed on the pixel signal obtained by the normal light observation. In addition, the special light observation may be infrared light observation in which infrared light is applied to the organ surface to view a deeper region than the organ surface, or multispectral observation utilizing hyperspectral spectroscopy.

### [Recording Device]

The recording device 5053 is a device for recording pixels acquired from the CCU 5039, and is, for example, a recorder. The recording device 5053 records the image acquired from the CCU 5039 on an HDD, an SDD, or an optical disc. The recording device 5053 may be connected to a network in the hospital and accessible from equipment outside the surgical room. In addition, the recording device 5053 may have an image down-conversion function or an image up-conversion function.

### [Display Device]

The display device 5041 is a device capable of displaying an image, and is, for example, a display monitor. The display device 5041 displays a display image based on the pixel signals subjected to image processing by the CCU 5039 under the control from the CCU 5039. It should be noted that the display device 5041 may include a camera and a microphone to function also as an input device that enables visual line recognition, voice recognition, and instruction input by gesture.

### [Output Device]

The output device 5055 is a device that outputs information acquired from the CCU 5039, and is, for example, a printer. The output device 5055 prints, for example, a print image based on the pixel signals acquired from the CCU 5039 on paper.

### [Support Device]

The support device 5027 is an articulated arm that includes a base part 5029 having an arm control device 5045, an arm part 5031 extending from the base part 5029, and a holding part 5032 attached to the distal end of the arm part 5031. The arm control device 5045 includes a processor such as a CPU and controls the driving of the arm part 5031 by operating according to a predetermined program. The support device 5027 controls, for example, the position and posture of the endoscope 5001 held by the holding part 5032 by controlling parameters such as the length of each link 5035 configuring the arm part 5031 and the rotation angle and the torque of each joint 5033 with the arm control device 5045. Accordingly, the endoscope 5001 can be changed to the desired position or posture, the scope 5003 can be inserted into the patient 5071, and the observation region in the body can be changed. The support device 5027 functions as an endoscope support arm that supports the endoscope 5001 during the surgery. Accordingly, the support device 5027 can take the role of a scopist who is an assistant holding the endoscope 5001. In addition, the support device 5027 may be a device that supports a microscope device 5301 to be described later, and can also be referred to as a medical support arm. It should be noted that the control of the support device 5027 may be an autonomous control method by the arm control device 5045, or a control method controlled by the arm control device 5045 on the basis of input by the user. For example, the control method may be a master/slave method in which the support device 5027 as a slave device (replica device) that is a patient cart is controlled on the basis of the movement of a master device (primary device) that is a practitioner console at hand of the user. In addition, the support device 5027 may be remotely controllable from the outside of the surgical room.

The example of the endoscope system 5000 to which the technique according to the present disclosure can be applied has been described above. For example, the technique according to the present disclosure may be applied to a microscope system.

### [Microscope Surgery System]

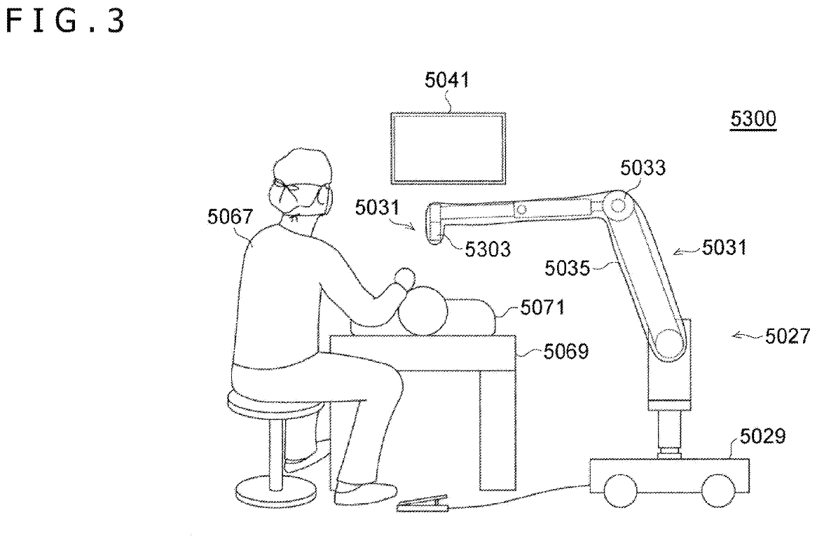

FIG. 3 is a diagram for depicting an example of a schematic configuration of a microscope surgery system to which the technique according to the present disclosure can be applied. It should be noted that, in the following description, the configurations similar to the endoscope system 5000 are denoted by the same symbols, and the duplicated descriptions thereof are omitted.

FIG. 3 schematically depicts a state in which the practitioner 5067 is performing surgery on the patient 5071 on the patient bed 5069 by using a microscope surgery system 5300. It should be noted that, for the sake of simplicity, FIG. 3 omits the illustration of the cart 5037 among the configurations of the microscope surgery system 5300, and illustrates the microscope device 5301 in place of the endoscope 5001 in a simplified manner. However, the microscope device 5301 in this description may refer to a microscope part 5303 provided at the distal end of the link 5035 or to the entire configuration including the microscope part 5303 and the support device 5027.

As depicted in FIG. 3, during surgery, an image of a surgery part photographed by the microscope device 5301 is enlarged and displayed on the display device 5041 installed in the surgical room by using the microscope surgery system 5300. The display device 5041 is installed at a position opposite to the practitioner 5067, and the practitioner 5067 performs various procedures, such as removal of an affected part, for example, on a surgery part while observing the state of the surgery part by the video reflected on the display device 5041. The microscope surgery system is used in, for example, ophthalmic surgery and brain surgery, but may be used in abdominal surgery and the like.

Each of the examples of the endoscope system 5000 and the microscope surgery system 5300 to which the technique according to the present disclosure can be applied has been described above. It should be noted that systems to which the technique according to the present disclosure can be applied are not limited to such examples. For example, the support device 5027 can support other observation devices or other surgical instruments at the distal end thereof, in place of the endoscope 5001 or the microscope part 5303. As other observation devices, for example, forceps, tweezers, pneumoperitoneum tubes for pneumoperitoneum, energy treatment tools that incise tissues or seal blood vessels by cauterization, or the like can be applied. By supporting these observation devices and surgical instruments with the support device, the position can be more stably fixed, and the burden on the medical staff can be reduced, as compared with a case in which the medical staff manually support. The technique according to the present disclosure may be applied to a support device that supports a configuration other than such a microscope part.

The technique according to the present disclosure can preferably be applied to the CCU 5039 among the configurations described above.

### [Functional Block of CCU 5039]

Hereinafter, an example of the endoscope system 5000 of FIG. 1 to which the present technique is applied will be described as a surgery system to which the present technique is applied.

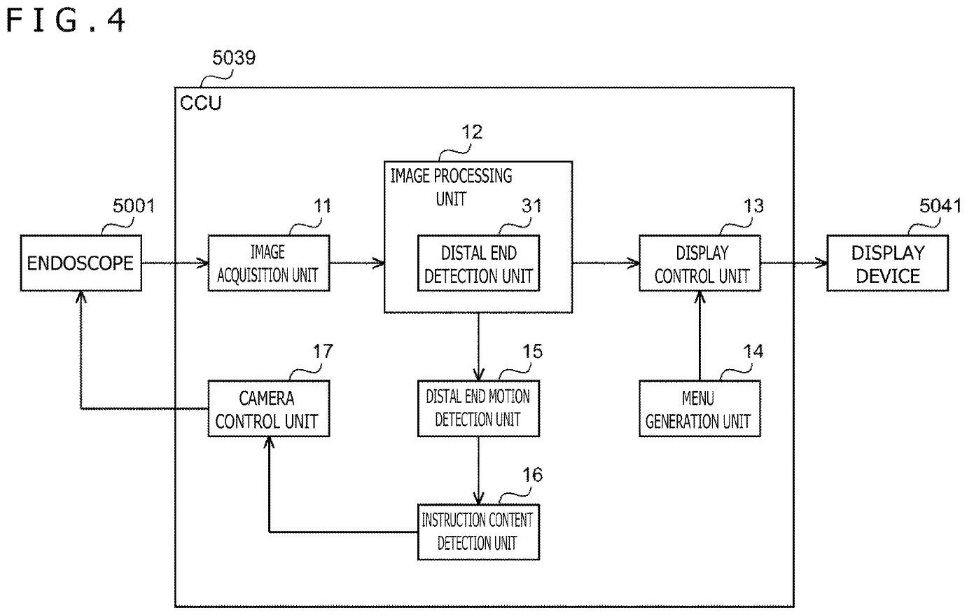

FIG. 4 is a diagram for depicting the CCU 5039 of FIG. 1 in functional blocks. It should be noted that, in the drawing, the parts in common to the endoscope system 5000 of FIG. 1 are denoted by the same symbols, and the descriptions thereof are omitted. In FIG. 4, functional blocks other than those related to the present technique are omitted.

In FIG. 4, the CCU 5039 has an image acquisition unit 11, an image processing unit 12, a display control unit 13, a menu generation unit 14, a distal end motion detection unit 15, an instruction content detection unit 16, and a camera control unit 17.

The image acquisition unit 11 acquires the pixel signals output from the endoscope 5001 and acquires a surgery image that is an image of the surgery region photographed by the endoscope 5001. It should be noted that the surgery region is not limited to the region where the surgery is actually performed by the practitioner, but includes the region observed by the endoscope 5001. The image acquisition unit 11 supplies the acquired surgery image to the image processing unit 12.

The image processing unit 12 performs image processing such as development processing (for example, demosaic processing) and correction processing on the surgery image from the image acquisition unit 11. The image processing unit 12 supplies the processed surgery image to the display control unit 13.

The image processing unit 12 includes a distal end detection unit 31. On the basis of the surgery image (or, the processed surgery image) from the image acquisition unit 11, the distal end detection unit 31 detects an image of a distal end part of an operation body reflected on the surgery image. The operation body means at least one of the surgical instrument and the hand. It should be noted that, in the endoscope system 5000, the hand of the practitioner is rarely reflected on the surgery image. However, the present technique can be applied to other surgery systems such as the microscope surgery system 5300 of FIG. 3. In other surgery systems, the hand of the practitioner can be reflected on the surgery image. Therefore, in consideration of the application of the present technique other than the endoscope system 5000, it is assumed that the hand can be included as the operation body reflected on the surgery image.

In a case where the operation body is a surgical instrument, the distal end part of the operation body refers to the range of a predetermined length including the distal end of the surgical instrument. In a case where the operation body is a hand, the distal end part of the operation body refers to the range of a predetermined length including the distal end (fingertip) of any finger. The surgical instrument is, for example, an arbitrary tool used in surgery, such as an energy device such as a freely selected scalpel or forceps.

When detecting the image of the distal end part of the operation body, the distal end detection unit 31 supplies the position of the distal end part of the operation body on the surgery image to the distal end motion detection unit 15.

The display control unit 13 generates a display image to be supplied to the display device 5041 on the basis of the surgery image from the image processing unit 12. The display image is, for example, an image including a part or the whole of the surgery image from the image processing unit 12. The display control unit 13 generates a display image in which an image of a virtual menu from the menu generation unit 14 is superimposed on the surgery image from the image processing unit 12. The display control unit 13 supplies the generated display image to the display device 5041.

The menu generation unit 14 generates an image of the virtual menu and supplies the same to the display control unit 13. The virtual menu is a menu that is virtually presented in the space of the surgery region, and represents a menu that depicts instruction contents that can be instructed by the practitioner regarding the control of the endoscope 5001 (medical imaging device), the CCU 5039 (control device), or the like. The details of the virtual menu will be described later.

On the basis of the position of the distal end part of the operation body from the distal end detection unit 31 of the image processing unit 12, the distal end motion detection unit 15 detects the motion (position change) of the distal end part of the operation body. The distal end motion detection unit 15 supplies the detected motion to the instruction content detection unit 16.

On the basis of the motion of the distal end part of the operation body from the distal end motion detection unit 15, the instruction content detection unit 16 detects the instruction content selected by the operation body among those that are presented by the virtual menu and can be selected (instructed). Accordingly, the instruction content detection unit 16 accepts an instruction operation by the operation body.

The instruction content detection unit 16 supplies the detected instruction content to the camera control unit 17 or the like. It should be noted that the instruction content selected by the operation body is not limited to one related to the control of the endoscope 5001. For example, the instruction content may be an instruction content related to the control of a freely selected device such as the CCU 5039 or the display device 5041. According to the detected instruction content, the instruction content detection unit 16 supplies the detected instruction content to even a processing unit or the like other than the camera control unit 17. In the following, however, it is assumed that the instruction content selected by the operation body relates to the control of the endoscope 5001, and the instruction content detected by the instruction content detection unit 16 is supplied to the camera control unit 17.

The camera control unit 17 controls the focus, the zoom, or the like of the endoscope 5001 on the basis of the instruction content supplied from the instruction content detection unit 16.

### [Detail of Processing of CCU 5039]

The details of processing in the CCU 5039 will be described.

[Operation on Virtual Menu with Use of Operation Body]

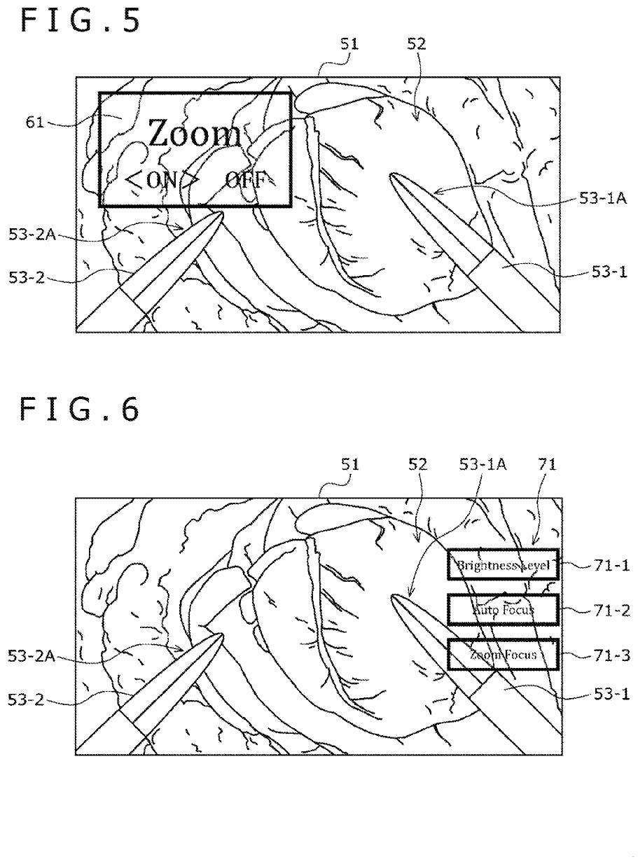

FIG. 5 is a diagram exemplifying a display image in which a virtual menu is displayed.

A display image 51 in FIG. 5 is an example of the display image supplied from the display control unit 13 to the display device 5041. The display image 51 includes a part or the whole of an image (surgery image) of a surgery region 52 photographed by the endoscope 5001. In the surgery region 52, there are organs and the like of the inside of the body of the patient 5071 and surgical instruments 53-1 and 53-2 such as forceps operated by the practitioner.

In a partial region of the display image 51, an image of a virtual menu 61 that is generated by the menu generation unit 14 and is superimposed on the surgery image in the display control unit 13 is displayed.

The virtual menu 61 is presented as a virtual translucent button (icon) in the space of the surgery region 52. When a predetermined operation is performed on the virtual menu 61 of FIG. 5 by the operation body (the surgical instrument or hand), the zoom control of the endoscope 5001 is switched from on to off or from off to on. Specifically, according to the display of the virtual menu 61, the current zoom state is on. Thus, when a predetermined operation is performed on the virtual menu 61 by the operation body, the zoom control of the endoscope 5001 is switched from on to off.

For example, it is assumed that the practitioner performs an operation of touching (coming into contact with) the virtual menu 61 by using a distal end part 53-1A of a surgical instrument 53-1, which is the operation body operated in the surgery region 52, or a distal end part 53-2A of a surgical instrument 53-2. The operation (motion) on the virtual menu 61 by the practitioner is detected by the distal end detection unit 31, the distal end motion detection unit 15, and the instruction content detection unit 16. Accordingly, an instruction operation to switch the zoom control from on to off, which is the instruction content corresponding to the virtual menu 61, is accepted by the instruction content detection unit 16. In this case, the camera control unit 17 controls the endoscope 5001 such that, for example, the focal distance of the camera is on the widest angle side.

According to the operation on the virtual menu 61 with use of such an operation body, the practitioner can intuitively and easily perform the instruction operation for the device even in a case where both hands of the practitioner are occupied by the surgical instruments or even in a case where the hands need to be kept clean.

That is, the practitioner performs surgery while viewing the surgery image photographed by the medical imaging device such the endoscope 5001 or the microscope device 5301 with the display device 5041. At this time, there is a case in which the practitioner grips the surgical instruments by both hands or a case in which the practitioner cannot touch medical equipment in an unclean region to keep the hands clean. In this case, it is difficult for the practitioner to directly operate the medical imaging device or other pieces of medical equipment (for example, an energy device used for removal of the surgery part). Therefore, it is general for assistants and nurses to operate the medical imaging device or other pieces of medical equipment. It is possible for the practitioner to operate the medical imaging device or other pieces of medical equipment by operating a footswitch connected to the medical equipment or by giving a voice instruction.

However, in order to perform more efficient surgery, it is preferable that it can be intuitively operated by the hand of the practitioner. In the present technique, the virtual menu is superimposed on the surgery image displayed on the display device 200, and the practitioner can operate the virtual menu by operating the operation body such as a surgical instrument used for the surgery. Therefore, even in a case where both hands of the practitioner are occupied by the surgical instruments or even in a case where the hands need to be kept clean, the practitioner can intuitively and easily perform the instruction operation for the device by the hands without touching the device in an unclean region.

[Detection of Operation on Virtual Menu with Use of Operation Body]

Next, detection of an operation on the virtual menu with use of the operation body will be described.

FIG. 6 is a diagram exemplifying a display image in which a virtual menu is displayed. It should be noted that the parts in common to the display image of FIG. 5 are denoted by the same symbols, and the descriptions thereof are omitted.

In the display image 51 of FIG. 6, an image of a virtual menu 71 that is generated by the menu generation unit 14 and is superimposed on the surgery image in the display control unit 13 is displayed. There are three kinds of instruction contents that can be selected (instructed) in the virtual menu 71. Since a case in which only one kind of instruction content can be selected is exemplified in the virtual menu 61 of FIG. 5, the virtual menu 71 of FIG. 6 differs from the virtual menu 61 of FIG. 5.

The virtual menu 71 has images of three buttons 71-1, 71-2, and 71-3. The button 71-1 corresponds to an instruction content related to the brightness (Brightness Level) of the surgery image (display image). The button 71-2 corresponds to an instruction content related to auto focus (Auto Focus). The button 71-3 corresponds to an instruction content related to zoom (Zoom Focus). The respective buttons 71-1, 71-2, and 71-3 of the virtual menu 71 are presented as virtual translucent buttons in the space of the surgery region 52.

The instruction content related to the brightness (Brightness Level) of the surgery image corresponding to the button 71-1 is, for example, an instruction to adjust the brightness. In a case where the button 71-1 is operated by the operation body, for example, a brightness adjustment menu is subsequently displayed as a virtual menu.

The instruction content related to the auto focus (Auto Focus) corresponding to the button 71-2 is, for example, an instruction to select a target to be focused by auto focus (AF). In a case where the button 71-2 is operated by the operation body, for example, an AF adjustment menu (AF selection screen) is subsequently displayed as a virtual menu. The instruction content related to AF may be, for example, an instruction (One Push AF) to execute a focus motion once by AF on the basis of the surgery image when the button 71-2 is operated by the operation body.

The instruction content related to the zoom (Zoom Focus) corresponding to the button 71-3 is, for example, an instruction to select a target to be zoomed (enlarged). In a case where the button 71-3 is operated by the operation body, for example, a zoom adjustment menu (zoom selection screen) is subsequently displayed as a virtual menu.

The distal end detection unit 31 detects images of the distal end part 53-1A of the surgical instrument 53-1 and the distal end part 53-2A of the surgical instrument 53-2 reflected on the surgery image from the endoscope 5001. On the basis of the positions of the images of the distal end part 53-1A and the distal end part 53-2A detected by the distal end detection unit 31, the distal end motion detection unit 15 detects the motions (position changes) of the distal end part 53-1A and the distal end part 53-2A.

The instruction content detection unit 16 recognizes the positions of the images of the respective buttons 71-1, 71-2, and 71-3 of the virtual menu 71 generated by the menu generation unit 14 on the surgery image. It is assumed that, on the basis of the motions of the distal end part 53-1A and the distal end part 53-2A detected by the distal end motion detection unit 15, the instruction content detection unit 16 detects that the distal end part 53-1A or the distal end part 53-2A has performed a predetermined operation on any one of the buttons 71-1, 71-2, and 71-3 of the virtual menu 71. In this case, the instruction content detection unit 16 detects the instruction content corresponding to the button on which the predetermined operation has been performed. That is, the instruction content detection unit 16 accepts the instruction operation of the instruction content corresponding to the button on which the predetermined operation has been performed by the operation body.

Here, the detection of the distal end parts 53-1A and 53-2A of the surgical instruments 53-1 and 53-2 by the distal end detection unit 31 may be performed by a machine learning method. For example, the distal end detection unit 31 detects the distal end part of the operation body (the surgical instrument and the finger) included in the surgery image by using instance segmentation in which object detection and image region division for each object are performed on the surgery image. That is, the image region of the operation body included in the surgery image is detected by using an inference model having a structure of a neural network such as a CNN (Convolutional Neural Network). The inference model is learned with a learning image in which the image region of the operation body is annotated (tagged). The detection of the operation body by the learning model may be detection of the distal end of the operation body or detection of a region having a predetermined length (for example, several centimeters) including the distal end of the operation body.

The conditions under which the instruction content detection unit 16 accepts an operation (operation that instructs a predetermined instruction content) on the virtual menu by the operation body will be described. First to fourth modes, which will be described next with reference to FIG. 6, differ from each other in the conditions under which an operation on the virtual menu by the operation body is accepted.

The first mode is as follows. The instruction content detection unit 16 determines whether or not an overlapping region has occurred between the image of the distal end part 53-1A of the surgical instrument 53-1 or the image of the distal end part 53-2A of the surgical instrument 53-2 (the image of the distal end part of the operation body), and the image of any one of the buttons 71-1, 71-2, and 71-3 of the virtual menu 71.

For example, the instruction content detection unit 16 can determine whether or not the overlapping region has occurred by any one of first to third determination conditions.

In the first determination condition, in a case where the image of the distal end part of the operation body and the image of the button of the virtual menu overlap each other by a certain area (preliminarily-fixed predetermined area) or more, it is determined that the overlapping region has occurred. In the second determination condition, in a case where the distal end of the image of the operation body is within the region of the image of the button of the virtual menu, it is determined that the overlapping region has occurred. In the third determination condition, in a case where both the first determination condition and the second determination condition are satisfied, it is determined that the overlapping region has occurred.

In a case where the instruction content detection unit 16 determines that the overlapping region has occurred between the image of the distal end part of the operation body and the image of any one of the buttons of the virtual menu 71, it is determined that the operation body has come into contact with the button. It should be noted that the button determined that the operation body has come into contact with is the one selected by the operation body.

In the first mode, when a predetermined button is selected by the operation body, the instruction content detection unit 16 accepts an instruction of the instruction content corresponding to the selected button. The instruction content detection unit 16 causes the camera control unit 17 or the like to execute control of the accepted instruction content.

Here, the touch (contact) of the distal end part of the operation body to the virtual menu may be detected with use of a sensor for ToF. For example, the sensor for ToF measures the depth of the surgery region. Accordingly, a region where the virtual menu is virtually presented and the position of the distal end part of the operation body are measured. Thus, it can be detected that the distal end part of the operation body has come into contact with any one of the buttons of the virtual menu. The touch (contact) of the distal end part of the operation body to the virtual menu may be detected by an SLAM (Simultaneous Localization and Mapping) technique with use of surgery images. On the basis of the distal end part of the operation body and the position of the virtual menu on a three-dimensional map, the SLAM technique can allow for detection of display of the virtual menu and the touch (contact) of the distal end part of the operation body to the virtual menu.

The second mode is as follows. As in the first mode, it is assumed that the instruction content detection unit 16 is in a state (hereinafter, referred to as a button selection state) in which a predetermined button has been selected by the operation body. It should be noted that the button selection state is canceled when there is no overlapping region between the image of the distal end part of the operation body and the image of the button.

It is assumed that, in the button selection state, the operation body has performed a reciprocating motion in the normal direction with respect to the plane of the selected button. This motion corresponds to the motion of pressing and releasing the button. In FIG. 6, the planes of the buttons 71-1, 71-2, and 71-3 are parallel to the screen of the display image 51. Thus, the normal direction with respect to the planes of the buttons 71-1, 71-2, and 71-3 is the direction of the optical axis of the endoscope 5001.

When the operation body performs the reciprocating motion in the normal direction with respect to the planes of the buttons, the instruction content detection unit 16 determines that an operation has been performed on the selected button and accepts an instruction of the instruction content corresponding to the selected button. The instruction content detection unit 16 causes the camera control unit 17 or the like to execute control of the accepted instruction content. It should be noted that the instruction content detection unit 16 may determine that an operation has been performed on the selected button in a case where the operation body has performed a motion in a direction (for example, a parallel direction) other than the normal direction with respect to the planes of the buttons.

The third mode is as follows. As in the first mode, it is assumed as the button selection state. In the button selection state, the instruction content detection unit 16 assumes that an operation or voice instructing a decision has been input by a predetermined operation of a footswitch connected directly or indirectly to the CCU 5039 or predetermined voice from a microphone (voice detection unit). At this time, the instruction content detection unit 16 determines that an operation has been performed on the selected button and accepts an instruction of the instruction content corresponding to the selected button. The instruction content detection unit 16 causes the camera control unit 17 or the like to execute control of the accepted instruction content.

That is, in the third mode, the button selection operation in the virtual menu is performed by the operation body, and the decision operation is performed by the physical motion of the practitioner. Accordingly, even in a case where the practitioner has moved the surgical instrument on the virtual menu with another intention, the decision operation cannot be performed. It should be noted that the decision operation may be performed by visual line input by the practitioner.

The fourth mode is as follows. As in the first mode, it is assumed as the button selection state. In a case where a period of time (for example, five seconds) equal to or larger than a threshold value which has been determined in advance has elapsed in the button selection state, the instruction content detection unit 16 determines that an operation has been performed on the selected button, and accepts an instruction of the instruction content corresponding to the selected button. The instruction content detection unit 16 causes the camera control unit 17 or the like to execute control of the accepted instruction content. It should be noted that the elapsed time from the start of the button selection state may be displayed being superimposed on the display image.

### [Processing Related to Display of Virtual Menu]

The processing related to the display of the virtual menus will be described.

The menu generation unit 14 generates an image of each button of the virtual menu as the virtual menu 71 in the display image 51 of FIG. 6. The menu generation unit 14 decides the position (superimposed region) where the image of each button of the virtual menu is superimposed on the display image. The menu generation unit 14 supplies the generated image of each button of the virtual menu and the position (superimposed region) where the image of each button is superimposed on the display image to the display control unit 13.

For the surgery image from the image processing unit 12, the display control unit 13 generates the display image 51 in which the image of each button of the virtual menu from the menu generation unit 14 is superimposed on the superimposed region from the menu generation unit 14.

The menu generation unit 14 defines the superimposed region where the image of each button of the virtual menu is superimposed as, for example, a region different from important regions in the display image. The important regions are the region where bleeding is occurring, the region of the organ, the region for surgery, and the region of a center part (center of the screen) of the display image. The center of the screen is the important region because the practitioner often displays the important region in the center of the screen.

The important regions such as the region where bleeding is occurring, the region of the organ, and the region for surgery are detected by the image processing unit 12 on the basis of the surgery image. The image processing unit 12 may detect the important regions in the surgery image by, for example, a machine learning method. The image processing unit 12 may detect not the important regions but non-important regions that are not the important regions. For example, since there is a high possibility that a region where the contrast in the surgery image is approximately constant is not the important region, the image processing unit 12 detects the region where the contrast is approximately constant as the non-important region. The image processing unit 12 supplies a region other than the non-important region, as the important region, to the menu generation unit 14.

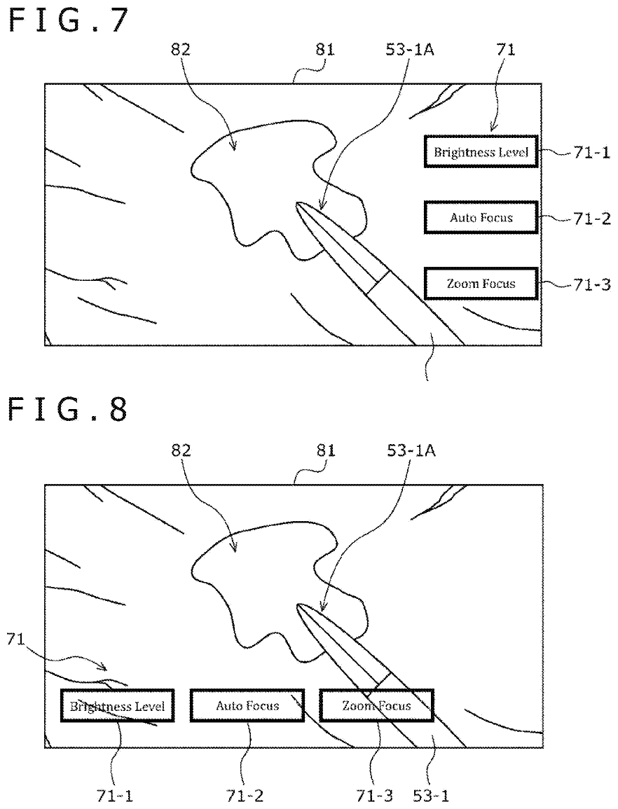

FIG. 7 and FIG. 8 are diagrams each exemplifying a display image in which the virtual menu is superimposed on the non-important region. Each of display images 81 in FIG. 7 and FIG. 8 includes a part or the whole of an image (surgery image) of a surgery region 82 photographed by the endoscope 5001. In each surgery region 82, there is a surgical instrument 53-1 such as forceps operated by the practitioner. It is assumed that the contrast is approximately constant in the right region of each display image 81. In this case, the right region of each display image 81 is detected as the non-important region. Accordingly, the right region of each display image 81 is defined as the superimposed region, and each of the buttons 71-1, 71-2, and 71-3 of the virtual menu 71 is superimposed on the right region of the display image 81 as depicted in FIG. 7.

It is assumed that a center part of each display image 81 is detected as, for example, the important region where bleeding is occurring. It is assumed that regions other than the center part are the non-important regions. In this case, a freely selected region other than the center part of each display image 81 can be the superimposed region. For example, each of the buttons 71-1, 71-2, and 71-3 of the virtual menu 71 is superimposed on the lower side of the display image 81 as depicted in FIG. 8.

It should be noted that, in the surgery image, any one or more of the region where bleeding is occurring, the region of the organ, the region for surgery, and the region of the center part (center of the screen) of the display image may be defined as the important regions. The menu generation unit 14 may define a region which is determined in advance for the display image as the superimposed region.

The display control unit 13 or the menu generation unit 14 may switch between a state in which the virtual menu is displayed on the display image and a state in which the virtual menu is deleted from the display image by voice input from a microphone. Accordingly, when the practitioner thinks the virtual menu superimposed on the display image is bothersome, the virtual menu can be instantly erased. On/off of the display of the virtual menu is not limited to a case of being performed on the basis of an input operation by the practitioner or the like. On/off of the display of the virtual menu may be performed on the basis of the states of other types of medical equipment. For example, in a case where an energy device is being used (in a case where an energy device is on), it is not necessary that the virtual menu is displayed. Accordingly, it is possible to prevent the virtual menu from hindering the surgery by the practitioner.

It is assumed that the instruction content detection unit 16 determines that the overlapping region has occurred between the image of the distal end part of the operation body and the image of any one of the buttons of the virtual menu, and the button selection state is started. In this case, the display control unit 13 or the menu generation unit 14 may change the transmissivity of the image of the selected button. For example, in a case where none of the buttons 71-1, 71-2, and 71-3 of the virtual menu 71 is in the button selection state in FIG. 8, the display control unit 13 or the menu generation unit 14 sets the transmissivities of the images of the buttons to approximately 80%. In a case where any one of the buttons 71-1, 71-2, and 71-3 of the virtual menu 71 is in the button selection state, the transmissivity of the image of the selected button is set to approximately 20%.

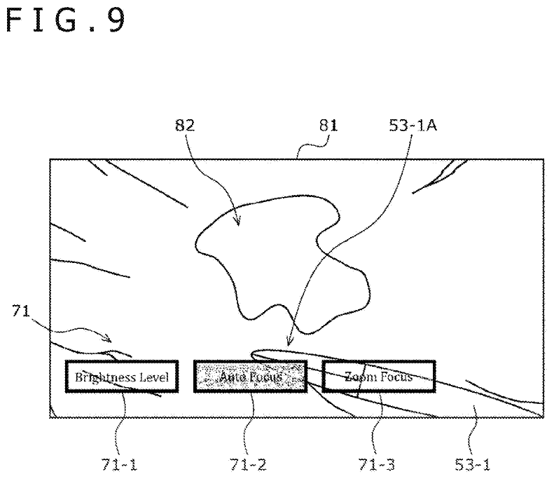

FIG. 9 is a diagram exemplifying a display image when a predetermined button of the virtual menu 71 of FIG. 8 is in the button selection state. In the drawing, the parts in common to FIG. 8 are denoted by the same symbols, and the descriptions thereof are omitted.

In FIG. 9, the distal end part 53-1A of the surgical instrument 53-1 comes into contact with the button 71-2 of the virtual menu 71, and the button 71-2 is in the button selection state. At this time, the transmissivity of the image of the button 71-2 is reduced from approximately 80%, which is not in the button selection state, to approximately 20%. Accordingly, the selected button 71-2 is easier to see and the button selection state is indicated to the practitioner. However, the transmissivity depicted here is an example, and the transmissivity is not limited to a specific value.

It should be noted that the instruction content corresponding to each button of the virtual menu is not limited to the switching between on and off of the predetermined control content, but may be the instruction content associated with various functions. For example, in a case where a button called AF associated with the AF selection mode is selected, a virtual AF selection screen may be presented in the space of the surgery region. In this case, when the practitioner uses the operation body to specify an AF point on the AF selection screen, the focus of the endoscope 5001 is controlled by using the specified AF point as the AF target region.

In a case where a predetermined button of the virtual menu is selected, the display control unit 13 generates, for example, a display image in which an image of a virtual slide bar is superimposed on the surgery image and outputs the same to the display device 5041. Accordingly, the virtual slide bar may be presented in the space of the surgery region. In this case, the degree corresponding to the predetermined control content, for example, the intensity of illumination light, the degree of zoom, or the like is changed by the practitioner operating the virtual slide bar with use of the operation body.

The virtual menu may be presented in a region that keeps a certain distance from an organ. In particular, in a case where the surgery image and the virtual menu are three-dimensionally displayed, the virtual menu may be automatically presented in the region that keeps a certain distance from the organ. Accordingly, a situation where the surgical instrument comes into contact with the organ can be reduced when the practitioner operates the virtual menu.

The user may be able to set and change the display mode of the virtual menu and the instruction content that can be instructed (selected) in the virtual menu. For example, the user may be able to select the presentation position of the virtual menu, the arrangement of the buttons, the instruction contents (types of functions) associated with the buttons, and the like among a plurality of candidates.

### [Processing Procedure of CCU 5039]

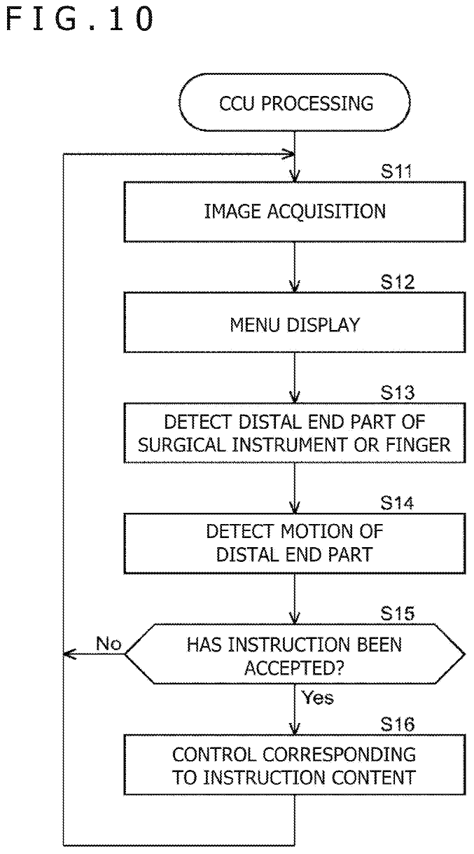

FIG. 10 is a flowchart exemplifying a procedure of processing related to the presentation of the virtual menu performed by the CCU 5039.

In Step S11, the image acquisition unit 11 acquires from the endoscope 5001 a surgery image in which a surgery region is photographed. The processing proceeds from Step S11 to Step S12.

In Step S12, the menu generation unit 14 generates an image of the virtual menu and supplies the same to the display control unit 13. The display control unit 13 generates a display image in which the image of the virtual menu from the menu generation unit 14 is superimposed on the surgery image acquired in Step S11 and supplies the same to the display device 5041. The processing proceeds from Step S12 to Step S13.

In Step S13, the distal end detection unit 31 detects an image of the distal end part of the operation body (the surgical instrument and the finger) reflected on the surgery image acquired in Step S11. The processing proceeds from Step S13 to Step S14.

In Step S14, the distal end motion detection unit 15 detects the motion (position change) from the past to the present of the distal end part detected in Step S13. The processing proceeds from Step S14 to Step S15.

In Step S15, the instruction content detection unit 16 determines, on the basis of the motion of the distal end part detected in Step S14, whether or not an instruction of a predetermined instruction content has been accepted by an operation of the distal end part of the operation body on the virtual menu.

In a case where it is determined in Step S15 that the instruction has not been accepted, the processing returns to Step S11 and is repeated from Step S11.

In a case where it is determined in Step S15 that the instruction has been accepted, the processing proceeds from Step S15 to Step S16.

In Step S16, the image processing unit 12, the display control unit 13, or the camera control unit 17 executes control corresponding to the instruction content accepted by the instruction content detection unit 16 in Step S15. The processing returns from Step S16 to Step S11 and is repeated from Step S11.

According to the above processing of the CCU 5039, the practitioner can perform the instruction operation of the predetermined instruction content related to the control of the devices such as the endoscope 5001 and the CCU 5039 on the virtual menu virtually presented to the surgery region by using the operation body of the surgery region or the hand. Therefore, even in a case where both hands of the practitioner are occupied by the surgical instruments or even in a case where the hands need to be kept clean, the practitioner can intuitively and easily perform the instruction operation for the device by the hand without touching the device in an unclean region.

### [Other Modes]

In a case where an instruction of shifting to a drawing mode (instruction mode) is given on the virtual menu, the display control unit 13 may generate a display image in which a virtual whiteboard (transmission type) enabling writing by using the operation body is superimposed on the surgery image and output the same to the display device 5041.

Instead of presenting the virtual menu, an instruction of a predetermined instruction content may be given only by an operation (motion) of the operation body. For example, in a case where the instruction content detection unit 16 detects a gesture of the distal end part of the operation body which has been determined in advance, an instruction of the instruction content corresponding to the gesture may be accepted. For example, in a case where the image of the distal end part of the operation body comes into contact with each of the upper, lower, left, and right end parts of the display image or a preliminarily-fixed region, the instruction content detection unit 16 may accept as an instruction of the instruction content preliminarily associated with each end part and each region.

### <Program>

Some or all of a series of processing in the CCU 5039 described above can be executed by hardware or software. In a case where the series of processing is executed by software, programs that configure the software are installed on a computer. Here, the computer includes a computer built into dedicated hardware, for example, a general-purpose personal computer that can execute various functions by installing various programs, and the like.

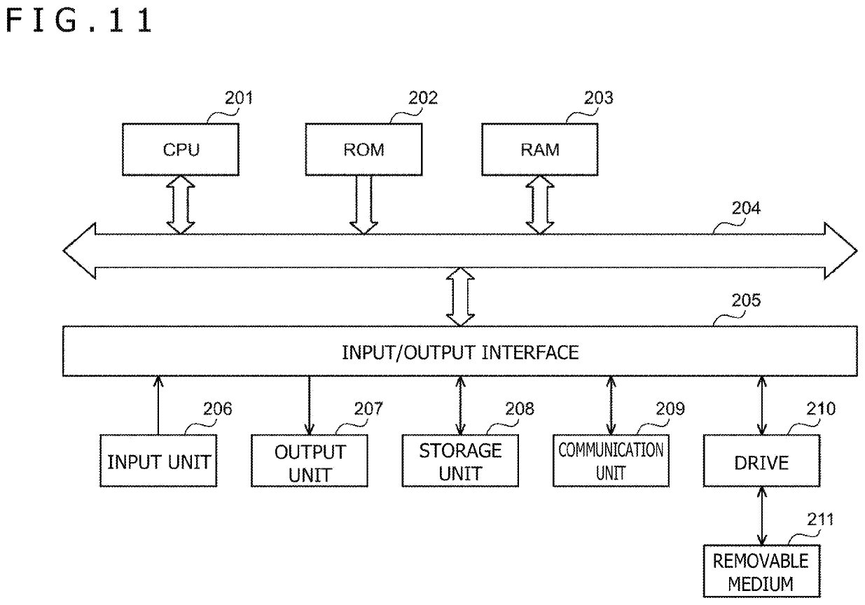

FIG. 11 is a block diagram for depicting a configuration example of hardware of a computer that executes a series of processing described above by a program.

In the computer, a CPU (Central Processing Unit) 201, a ROM (Read Only Memory) 202, and a RAM (Random Access Memory) 203 are connected to each other by a bus 204.

An input/output interface 205 is further connected to the bus 204. An input unit 206, an output unit 207, a storage unit 208, a communication unit 209, and a drive 210 are connected to the input/output interface 205.

The input unit 206 includes a keyboard, a mouse, a microphone, and the like. The output unit 207 includes a display, a speaker, and the like. The storage unit 208 includes a hard disk, a nonvolatile memory, and the like. The communication unit 209 includes a network interface, and the like. The drive 210 drives a removable medium 211 such as a magnetic disk, an optical disc, a magneto-optical disk, or a semiconductor memory.

In the computer configured as described above, the series of processing described above is performed when the CPU 201 loads, for example, a program stored in the storage unit 208 into the RAM 203 via the input/output interface 205 and the bus 204 and executes the same.

The program executed by the computer (CPU 201) can be provided by being recorded on the removable medium 211 as, for example, a package medium or the like. In addition, the program can be provided via a wired or wireless transmission medium such as a local area network, the Internet, or digital satellite broadcasting.

In the computer, the program can be installed in the storage unit 208 via the input/output interface 205 by loading the removable medium 211 into the drive 210. In addition, the program can be received by the communication unit 209 and installed in the storage unit 208 via a wired or wireless transmission medium. Moreover, the program can be pre-installed in the ROM 202 or the storage unit 208.

It should be noted that the program executed by the computer may be a program by which processing is performed in time series according to the order described in this specification, or a program by which processing is performed in parallel or at a necessary timing such as when a call is made.

The present technique can also be configured as follows.

(1)

A surgery system including:
a medical imaging device that captures a surgery region to acquire a surgery image that is an image of the surgery region; and
a control device that outputs a display image to a display device on the basis of the surgery image acquired by the medical imaging device,
in which, with at least one of a surgical instrument and a hand reflected on the surgery image as an operation body, the control device accepts a predetermined instruction operation by the operation body related to control of the medical imaging device or the control device on the basis of an image of the operation body in the surgery image.
   (2)

The surgery system according to (1) above,
in which the instruction operation includes an operation for selecting any one of a plurality of kinds of instruction contents related to the control.
   (3)

The surgery system according to (1) or (2) above,
in which the instruction operation includes an operation for giving an instruction to switch the content of the control.
   (4)

The surgery system according to any one of (1) to (3) above,
in which the control device generates the display image in which an image of a virtual menu which is virtually presented in a space of the surgery region and represents instruction contents that is able to be instructed with respect to the control is superimposed on the surgery image.
   (5)

The surgery system according to (4) above,
in which the control device accepts an operation of the operation body on the virtual menu as the instruction operation.
   (6)

The surgery system according to (4) or (5) above,
in which the control device accepts the instruction operation in a case where the operation body comes into contact with the virtual menu.
   (7)

The surgery system according to (6),
in which the control device detects an image of a distal end part of the operation body reflected on the surgery image and detects contact of the operation body with the virtual menu on the basis of a position of the image of the distal end part in the surgery image.
   (8)

The surgery system according to (6) or (7) above,
in which the control device detects an image of a distal end part of the operation body reflected on the surgery image and detects contact of the operation body with the virtual menu on the basis of an overlapping region between a position of the image of the distal end part in the surgery image and a position of the image of the virtual menu in the surgery image.
   (9)

The surgery system according to (7) or (8) above,
in which the distal end part of the operation body includes a distal end part of the surgical instrument or a distal end part of a finger of the hand.
   (10)

The surgery system according to (8) above,
in which the control device accepts the instruction operation in a case where the overlapping region has occurred and a motion of the operation body in a predetermined direction on the virtual menu has been detected.
   (11)

The surgery system according to (10) above,
in which the virtual menu has a plane with which the operation body is able to come into contact, and
the motion of the operation body in the predetermined direction includes a reciprocating motion in the normal direction of the plane.
   (12)

The surgery system according to (8) above,
in which the control device accepts the instruction operation in a case where the overlapping region has occurred and a period of time equal to or larger than a predetermined threshold value that is determined in advance has elapsed.
   (13)

The surgery system according to any one of (4) to (12) above,
in which the control device superimposes the image of the virtual menu on a region different from an important region in the surgery in the surgery image.
   (14)

The surgery system according to (13) above,
in which the important region includes any one or more of a region where bleeding is occurring, a region of an organ, a region for surgery, and a region of a center part of the display image.
   (15)

The surgery system according to any one of (4) to (14) above,
in which a voice input unit for detecting voice is provided, and
the control device switches presence or absence of presentation of the virtual menu on the basis of voice from the voice input unit.
   (16)

The surgery system according to any one of (4) to (15) above,
in which the control device does not present the virtual menu while an energy device is being used.
   (17)

The surgery system according to any one of (4) to (16) above, in which the virtual menu has a button for each instruction content that is able to be instructed.

(18)

The surgery system according to any one of (1) to (17) above, in which, in a case where an instruction to switch to an autofocus mode is accepted, the control device generates the display image in which a selection image for selecting a target region for autofocus is superimposed on the surgery image.

(19)

The surgery system according to any one of (1) to (18) above, in which, in a case where an instruction to switch to a drawing mode is accepted, the control device generates the display image in which a virtual whiteboard enabling writing with use of the operation body is superimposed on the surgery image.

(20)

The surgery system according to any one of (1) to (19) above, in which, in a case where an instruction of a predetermined instruction content is accepted, the control device generates the display image in which an image of a virtual slide bar which is virtually presented in the space of the surgery region and instructs a degree corresponding to a control content by the operation body is superimposed on the surgery image.

(21)

The surgery system according to any one of (4) to (20) above,
in which the control device presents the virtual menu at a position that is away from an organ by a predetermined distance.
   (22)

A surgery control device including:
a processing unit that accepts, with at least one of a surgical instrument and a hand reflected on a surgery image obtained by photographing a surgery region as an operation body, a predetermined instruction operation by the operation body on the basis of an image of the operation body in the surgery image.
   (23)

A control method,
in which a surgery control device has a processing unit and the processing unit accepts, with at least one of a surgical instrument and a hand reflected on a surgery image obtained by photographing a surgery region as an operation body, a predetermined instruction operation by the operation body on the basis of an image of the operation body in the surgery image.
   (24)

A program that causes a computer to function as a processing unit that accepts, with at least one of a surgical instrument and a hand reflected on a surgery image obtained by photographing a surgery region as an operation body, a predetermined instruction operation by the operation body on the basis of an image of the operation body.

### REFERENCES SIGNS LIST

11: Image acquisition unit
12: Image processing unit
13: Display control unit
14: Menu generation unit
15: Distal end motion detection unit
16: Instruction content detection unit
17: Camera control unit
31: Distal end detection unit
5000: Endoscope system
5001: Endoscope
5039: CCU
5300: Microscope surgery system

1. A surgery system comprising:
a medical imaging device that captures a surgery region to acquire a surgery image that is an image of the surgery region; and
a control device that outputs a display image to a display device on a basis of the surgery image acquired by the medical imaging device,
wherein, with at least one of a surgical instrument and a hand reflected on the surgery image as an operation body, the control device accepts a predetermined instruction operation by the operation body related to control of the medical imaging device or the control device on a basis of an image of the operation body in the surgery image.

2. The surgery system according to claim 1,
wherein the instruction operation includes an operation for selecting any one of a plurality of kinds of instruction contents related to the control.

3. The surgery system according to claim 1,
wherein the instruction operation includes an operation for giving an instruction to switch the content of the control.

4. The surgery system according to claim 1,
wherein the control device generates the display image in which an image of a virtual menu which is virtually presented in a space of the surgery region and represents instruction contents that is able to be instructed with respect to the control is superimposed on the surgery image.

5. The surgery system according to claim 4,
wherein the control device accepts an operation of the operation body on the virtual menu as the instruction operation.

6. The surgery system according to claim 4,
wherein the control device accepts the instruction operation in a case where the operation body comes into contact with the virtual menu.

7. The surgery system according to claim 6,
wherein the control device detects an image of a distal end part of the operation body reflected on the surgery image and detects contact of the operation body with the virtual menu on a basis of a position of the image of the distal end part in the surgery image.

8. The surgery system according to claim 6,
wherein the control device detects an image of a distal end part of the operation body reflected on the surgery image and detects contact of the operation body with the virtual menu on a basis of an overlapping region between a position of the image of the distal end part in the surgery image and a position of the image of the virtual menu in the surgery image.

9. The surgery system according to claim 8,
wherein the distal end part of the operation body includes a distal end part of the surgical instrument or a distal end part of a finger of the hand.

10. The surgery system according to claim 8,
wherein the control device accepts the instruction operation in a case where the overlapping region has occurred and a motion of the operation body in a predetermined direction on the virtual menu has been detected.

11. The surgery system according to claim 10,
wherein the virtual menu has a plane with which the operation body is able to come into contact, and
the motion of the operation body in the predetermined direction includes a reciprocating motion in the normal direction of the plane.

12. The surgery system according to claim 8,
wherein the control device accepts the instruction operation in a case where the overlapping region has occurred and a period of time equal to or larger than a predetermined threshold value that is determined in advance has elapsed.

13. The surgery system according to claim 4,
wherein the control device superimposes the image of the virtual menu on a region different from an important region in the surgery in the surgery image.

14. The surgery system according to claim 13,
wherein the important region includes any one or more of a region where bleeding is occurring, a region of an organ, a region for surgery, and a region of a center part of the display image.

15. The surgery system according to claim 4,
wherein a voice input unit for detecting voice is provided, and
the control device switches presence or absence of presentation of the virtual menu on a basis of voice from the voice input unit.

16. The surgery system according to claim 4,
wherein the control device does not present the virtual menu while an energy device is being used.

17. The surgery system according to claim 4,
wherein the control device presents the virtual menu at a position that is away from an organ by a predetermined distance.

18. A surgery control device comprising:
a processing unit that accepts, with at least one of a surgical instrument and a hand reflected on a surgery image obtained by photographing a surgery region as an operation body, a predetermined instruction operation by the operation body on a basis of an image of the operation body in the surgery image.

19. A control method,
wherein a surgery control device has a processing unit and the processing unit accepts, with at least one of a surgical instrument and a hand reflected on a surgery image obtained by photographing a surgery region as an operation body, a predetermined instruction operation by the operation body on a basis of an image of the operation body in the surgery image.

20. A program that causes a computer to function as a processing unit that accepts, with at least one of a surgical instrument and a hand reflected on a surgery image obtained by photographing a surgery region as an operation body, a predetermined instruction operation by the operation body on a basis of an image of the operation body.
